(19) United States
(12) Patent Application Publication
(10) Pub. No.: US 2010/0105898 A1
(43) Pub. Date: Apr. 29, 2010

(54) METHOD FOR PRODUCING SCOPINIUM SALTS

(75) Inventors: Joerg Brandenburg, Ingelheim am Rhein (DE); Werner Belzer, Ingelheim am Rhein (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM PHARMA GMBH & CO. KG, Ingelheim am Rhein (DE)

(21) Appl. No.: 12/524,630

(22) PCT Filed: Jan. 28, 2008
(86) PCT No.: PCT/EP2008/050988

§ 371 Date: Jul. 27, 2009

(30) Foreign Application Priority Data
Jan. 29, 2007 (EP) 07101363.5

(51) Int. Cl. C07D 498/08

(52) U.S. Cl. 544/105

(57) ABSTRACT

The invention relates to a new method of preparing scopinium salts of general formula 1

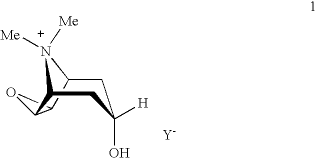

wherein Y⁻ may have the meanings given in the claims and in the specification.

The invention relates to a new method of preparing scopinium salts of general formula 1

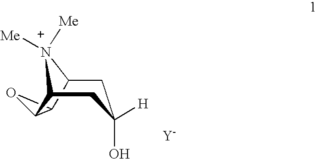

wherein Y⁻ may have the meanings given in the claims and in the specification.

### DESCRIPTION OF THE INVENTION

The present invention relates to a method of preparing scopinium salts of formula 1

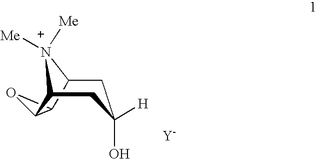

wherein
Y⁻ denotes a lipophilic anion with a single negative charge, preferably an anion selected from among hexafluorophosphate, tetrafluoroboranate, tetraphenylboranate and saccharinate, particularly preferably hexafluorophosphate or tetraphenylboranate
   characterised in that a compound of formula 2

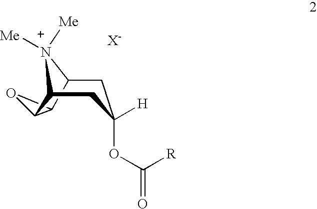

wherein
X⁻ denotes an anion with a single negative charge selected from among chloride, bromide, iodide, methanesulphonate, p-toluenesulphonate, nitrate and trifluoromethanesulphonate, preferably chloride, bromide, iodide, methanesulphonate, nitrate or trifluoromethanesulphonate, particularly preferably chloride, bromide or methanesulphonate, particularly preferably bromide; and
R denotes a group selected from C₁-C₄-alkyl, C₂-C₆-alkenyl and C₁-C₄-alkylene-phenyl, which may be substituted in each case by hydroxy, hydroxymethyl or C₁-C₄-alkoxy,
   optionally in the form of the solvates or hydrates thereof, is saponified in a suitable solvent with the addition of a suitable base to form initially a compound of formula 3

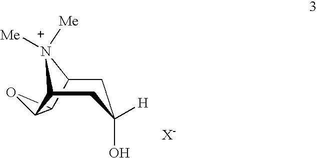

wherein X⁻ may have the meanings given above, and the compound of formula 3 is converted, without being isolated, into the compound of formula 1 by reaction with a salt Kat⁺Y⁻, where Kat⁺ denotes a cation selected from among Li⁺, Na⁺, K⁺, Mg²⁺, Ca²⁺, and Y⁻ may have the meanings given above.

A particularly preferred method according to the invention is characterised in that the reaction is carried out with a compound of formula 2, wherein
R denotes a group selected from —CH₃, —CH₂—CH₃, —CH₂—CH₂—OH, —CH(OH)—CH₃, —CH₂-phenyl, —CH(OH)-phenyl and —CH(CH₂OH)-phenyl, preferably —CH₃, —CH₂—CH₃, —CH₂-phenyl, and —CH(CH₂OH)-phenyl, particularly preferably —CH(CH₂OH)-phenyl.

A particularly preferred method is carried out with a compound of formula 2, wherein X⁻ denotes bromide and R denotes —CH(CH₂OH)-phenyl.

A particularly preferred method according to the invention relates to the preparation of a compound of formula 1 wherein
Y⁻ denotes an anion with a single negative charge selected from among hexafluorophosphate, tetrafluoroboranate and tetraphenylboranate, preferably hexafluorophosphate.

A particularly preferred method according to the invention is characterised in that the reaction of the compound of formula 2 to obtain the compound of formula 1 is carried out using a salt KatY, where Kat⁺ is selected from among Li⁺, Na⁺ and K⁺, particularly preferably Na⁺ and K⁺, and wherein Y⁻ may have the meanings given above.

The process according to the invention is characterised inter alia in that it allows direct access to salts of formula 1 from compounds of formula 2 in a single step without the need to isolate the intermediate compound of formula 3.

Examples of alkyl groups, as well as alkyl groups which are a part of other groups, include branched and unbranched alkyl groups with 1 to 4 carbon atoms. These include: methyl, ethyl, propyl, butyl. Unless stated otherwise, the above-mentioned designations propyl and butyl include all the possible isomeric forms. For example, the term propyl includes the two isomeric groups n-propyl and iso-propyl, the term butyl includes n-butyl, iso-butyl, sec. Butyl and tert.-butyl.

Examples of alkoxy or alkyloxy groups are branched and unbranched alkyl groups with 1 to 4 carbon atoms which are linked by an oxygen atom. These include: methoxy, ethoxy, propoxy, butoxy, for example. Unless stated otherwise, the above-mentioned designations include all the possible isomeric forms.

By lipophilic anions are meant according to the invention those anions the sodium or potassium salts of which have a solubility in polar organic solvents such as methanol or acetone of>1 wt. %.

The solvents used to carry out the process according to the invention are preferably polar solvents. Preferred solvents are selected according to the invention from among water, methanol, ethanol, propanol and isopropanol, while water and methanol are of exceptional importance according to the invention.

The bases used to saponify the compounds of formula 2 to form the compounds of formula 3 are preferably inorganic bases. Examples include the alkali or alkaline earth metal carbonates, hydroxides and alkoxides. The carbonates, hydroxides and alkoxides are preferably used in the form of their lithium, sodium or potassium salts. Preferred bases are selected from among sodium carbonate, lithium carbonate, potassium carbonate, calcium carbonate, sodium hydroxide, potassium hydroxide, sodium methoxide, sodium ethoxide, potassium methoxide or potassium ethoxide. Particularly preferably, one of the above-mentioned potassium or sodium salts is used as the inorganic base, while the use of potassium hydroxide or sodium methoxide is particularly preferred according to the invention.

Theoretically, equimolar amounts of base may preferably be used per mol of the compound of formula 2 used, while the base may optionally also be used in a slight excess. If methanol is used as the solvent, less base can be used per mol of the compound of formula 2 used. In such a case, the reaction is also carried out for example using 0.01 to 0.5 mol, preferably 0.02 to 0.3 mol, particularly preferably 0.04 to 0.15 mol of base per mol of the compound of formula 2 used.

According to the invention preferably 1 mol, more preferably 1-1.5 mol, optionally also 2-5 mol of the salt Kat⁺Y⁻ are used per mol of the compound of formula 2 used. It is apparent to the skilled man that the use of smaller amounts of salt Kat⁺Y⁻ is possible, but that this may then lead to only a partial reaction of the compound of formula 2. The salts Kat⁺Y⁻ are optionally also referred to only as salts KatY within the scope of the present invention.

The process according to the invention is preferably carried out under mild reaction conditions, i.e. at temperatures in the range from 10-55° C., particularly preferably 15-50° C., particularly preferably 20-45° C. After all the salts KatY have been added, and to some extent even during their addition, the compounds of formula 1 crystallise out from the solution. The products obtained may, if necessary, be purified by recrystallisation from one of the above-mentioned solvents. The crystals obtained are isolated and dried in vacuo.

The salts of quaternary ammonium compounds, such as for example those of formula 2 or 3, are generally readily soluble in water and alcohol. However, they are extremely poorly soluble in less polar organic solvents such as for example acetone, acetonitrile, hydrocarbons, halohydrocarbons or ethers. Chemical reactions with quaternary ammonium compounds are therefore limited in principle to reactions in water, alcohol or strongly polar aprotic solvents such as DMF or NMP. This gives rise to severe restrictions as to the choice of reactants or their separation from the target product.

Many synthesis strategies fail as a result of the impossibility or difficulty of separating quaternary ammonium compounds in aqueous or alcoholic solutions from other reaction components. This problem can be solved using the anions of formula 1. The selective precipitation or crystallisation of the quaternary ammonium compounds of formula 1 from alcohols or water may be carried out by reacting the compounds 2 with the corresponding salts KatY and in this way they can be isolated and purified with a regularly high yield.

By virtue of their very good solubility and the exceptionally high stability of the anion, the compounds 1 make it possible to carry out a range of reactions in less polar aprotic solvents and may be used wherever water or alcohol creates a problem. The synthesis of tiotropium salts of formula 4 illustrated in the following Scheme 1 and also described in detail in the experimental section of the present invention may serve as an example of this.

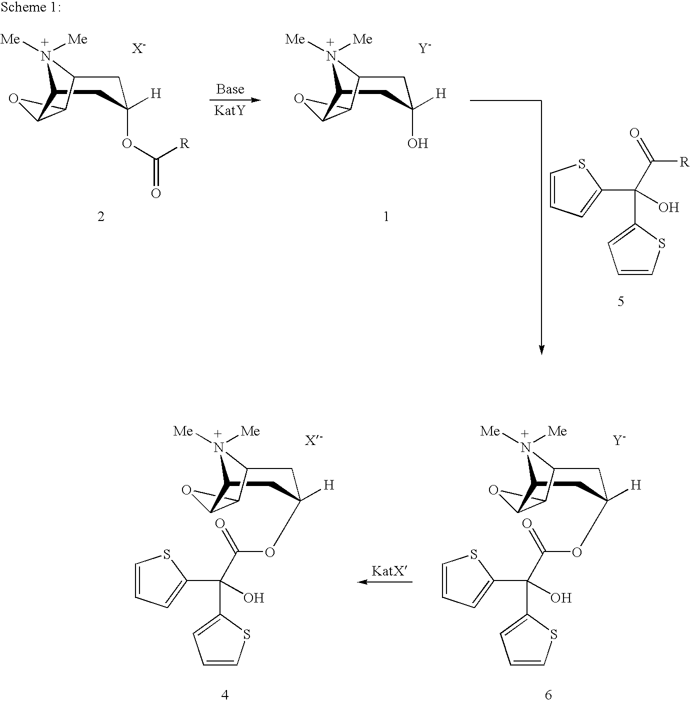

The present invention also relates to a method of preparing tiotropium salts of formula 4

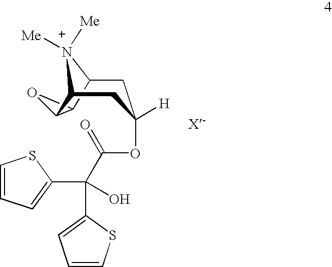

wherein
X′⁻ may denote an anion with a single negative charge, preferably an anion selected from among chloride, bromide, iodide, sulphate, phosphate, methanesulphonate, nitrate, maleate, acetate, citrate, fumarate, tartrate, oxalate, succinate, benzoate, p-toluenesulphonate and trifluoromethanesulphonate,
   characterised in that a compound of formula 2

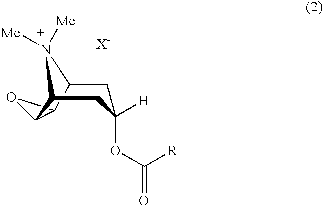

wherein
X⁻ denotes an anion with a single negative charge selected from among chloride, bromide, iodide, methanesulphonate, p-toluenesulphonate and trifluoromethanesulphonate, preferably chloride, bromide, iodide, methanesulphonate or trifluoromethanesulphonate, particularly preferably chloride, bromide or methanesulphonate, particularly preferably bromide; and
R denotes a group selected from C₁-C₄-alkyl, C₂-C₆-alkenyl and C₁-C₄-alkylene-phenyl, which may be substituted in each case by hydroxy, hydroxymethyl or C₁-C₄-alkoxy,
   optionally in the form of the acid addition salts thereof and optionally in the form of the hydrates thereof, is saponified in a suitable solvent with the addition of a suitable base to form initially a compound of formula 3

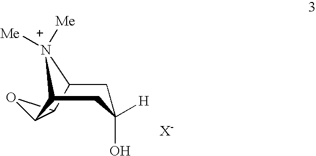

wherein X⁻ may have the meanings given above, and the compound of formula 3 without being isolated is converted by reaction with a salt Kat⁺Y⁻, wherein Kat⁺ denotes a cation selected from among Li⁺, Na⁺, K⁺, Mg²⁺, Ca²⁺, and
Y⁻ may denote a lipophilic anion with a single negative charge, preferably an anion selected from among hexafluorophosphate, tetrafluoroboranate, tetraphenylboranate and saccharinate, particularly preferably hexafluorophosphate or tetraphenylboranate
   into the compound of formula 1

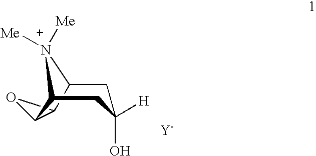

wherein Y⁻ may have the meanings given above,
and then the compound of formula 1 is reacted in one step with a compound of formula 5

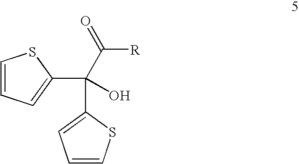

wherein
R denotes a group selected from among methoxy, ethoxy, propoxy, isopropoxy, isopropenyloxy, butoxy, O-N-succinimide, O-N-phthalimide, phenyloxy, nitrophenyloxy, fluorophenyloxy, pentafluorophenyloxy, vinyloxy, 2-allyloxy, -S-methyl, -S-ethyl and -S-phenyl,
   in a suitable solvent to form a compound of formula 6

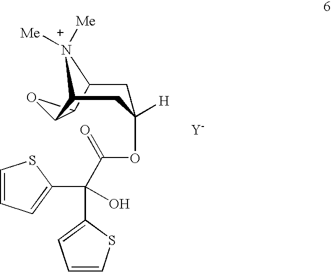

wherein the group Y⁻ may have the meanings given above, and the compound of formula 6 without being isolated is converted into the compound of formula 4 by reaction with a salt Kat⁺X′⁻, where Kat⁺ denotes a cation selected from among Li⁺, Na⁺, K⁺, Mg²⁺, Ca²⁺, organic cations with quaternary N (e.g. N,N-dialkylimidazolium, tetraalkylammonium) and X′⁻ may have the meanings given above.

Preferably the present invention relates to a method of preparing tiotropium salts of formula 4 , wherein
X^{′}⁻ may denote an anion with a single negative charge selected from among chloride, bromide, iodide, methanesulphonate, p-toluenesulphonate and trifluoromethanesulphonate, preferably chloride, bromide, iodide, methanesulphonate or trifluoromethanesulphonate, particularly preferably chloride, bromide or methanesulphonate, particularly preferably bromide.

A particularly preferred method according to the invention is characterised in that the reaction is carried out with a compound of formula 5, wherein
R denotes a group selected from among methoxy, ethoxy, propoxy, isopropoxy, isopropenyloxy, butoxy, O-N-succinimide, O-N-phthalimide, phenyloxy, nitrophenyloxy, fluorophenyloxy, pentafluorophenyloxy, vinyloxy and 2-allyloxy.

A particularly preferred method according to the invention is characterised in that the reaction is carried out with a compound of formula 5, wherein
R denotes a group selected from among methoxy, ethoxy, propoxy, isopropoxy, isopropenyloxy, butoxy, O-N-succinimide, O-N-phthalimide, vinyloxy and 2-allyloxy, preferably selected from methoxy, ethoxy, propoxy, and butoxy, particularly preferably methoxy or ethoxy.

A particularly preferred method according to the invention is characterised in that the reaction is carried out with a compound of formula 1, wherein
Y⁻ denotes an anion with a single negative charge selected from among hexafluorophosphate, tetrafluoroboranate and tetraphenylboranate, preferably hexafluorophosphate.

A particularly preferred method according to the invention is characterised in that that the final reaction of the compound of formula 6 to form the compound of formula 4 is carried out using a salt KatX′, wherein Kat⁺ is selected from among Li⁺, Na⁺, K⁺, Mg²⁺, Ca²⁺, organic cations with quaternary N (e.g. N,N-dialkylimidazolium, tetraalkylammonium) and wherein X′⁻ may have the meanings given above.

The process according to the invention is particularly characterised in that because of the solubility of the intermediates of formula 1 and 6 it can be carried out in relatively non-polar solvents. This allows the reaction to be carried out under very mild conditions which in the case of the sensitive tiotropium salts result in fewer side reactions and consequently a higher yield compared with reactions in highly polar aprotic solvents.

The reaction of the compounds of formula 1 with the compounds of formula 5 is preferably carried out in an aprotic organic solvent, preferably in a weakly polar organic solvent. Solvents which may be used according to the invention are particularly preferably acetone, pyridine, acetonitrile and methylethylketone, while acetone, acetonitrile and pyridine are most preferred. The reaction is particularly preferably carried out in a solvent selected from among acetone and acetonitrile, while the use of acetone is particularly preferred according to the invention.

It may in some cases make sense to activate the reaction of the compound of formula 1 with 5 by adding a catalyst. A particularly gentle activation is possible according to the invention using catalysts selected from among zeolites, lipases, tert. amines, such as for example N,N-dialkylamino-pyridine, 1,4-diazabicyclo[2,2,2]octane (DABCO) and diisopropylethylamine and alkoxides, such as for example, sodium or potassium-tert.butoxide, sodium or potassium isopropoxide or sodium or potassium ethoxide, while the use of zeolites and particularly zeolites and potassium-tert.-butoxide is particularly preferred, according to the invention. Particularly preferred zeolites are molecular sieves which are selected from among the molecular sieves of a basic nature consisting of sodium- or potassium-containing aluminosilicates, preferably molecular sieves with the empirical formula Na₁₂[(AlO₂)₁₂(SiO₂)₁₂]×H₂O, while the use of molecular sieve type 4 Å (indicating a pore size of 4 Angstrom) is particularly preferred according to the invention.

The reaction of 1 with 5 to form the compound of formula 6 may be carried out at elevated temperature, depending on the type of catalyst. Preferably, the reaction is carried out at a temperature of 30° C., particularly preferably within the range from 0 to 30° C.

The compounds of formula 5 may be obtained by methods known in the art. Mention may be made for example to WO03/057694, the contents of which are incorporated herein by reference.

In another aspect, the present invention relates to the use of compounds of formula 2 as starting compounds for preparing compounds of formula 4. In another aspect, the present invention relates to the use of compounds of formula 2 as starting compounds for preparing compounds of formula 6.

In another aspect, the present invention relates to a method of preparing compounds of formula 4, characterised in that a compound of formula 2 is used as a starting compound for preparing compounds of formula 4. In another aspect, the present invention relates to a method of preparing compounds of formula 6, characterised in that a compound of formula 2 is used as a starting compound for preparing compounds of formula 6.

The following Examples serve to illustrate methods of synthesis carried out by way of example. They are to be understood only as possible methods described by way of example without restricting the invention to their contents.

### EXAMPLE 1

### N-methylscopinium hexafluorophosphate

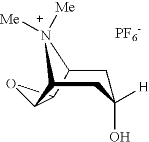

### Variant 1 a:

N-methylscopolaminium bromide is dissolved in water and saponified with the addition of an equimolar amount of potassium hydroxide solution at ambient temperature and combined with an equimolar amount or molar excess of a water-soluble hexafluorophosphate (sodium or potassium salt). The N-methylscopinium hexafluorophosphate crystallises out as a white, poorly water-soluble product, is isolated, optionally washed with methanol and then dried at approx. 40° C. in vacuo.

### Variant 1 b:

N-methylscopolaminium bromide (40 g) is dissolved in methanol (120 ml) and brought to a transesterification reaction by the addition of a catalytic amount (4-14 mol %) sodium methoxide or NaOH or conc. sodium hydroxide solution (20-45° C.) and then combined with an equimolar amount or molar excess of a solution of sodium hexafluorophosphate (18 g in 40 ml) methanol.

The N-methylscopinium hexafluorophosphate is precipitated/crystallises out as a white product which is poorly soluble in water, then it is isolated, optionally washed with methanol and then dried at approx. 40° C. in vacuo.

Yield: 88-95%

M.p.: 265-267° C. (melts with discoloration);

H-NMR: in acetonitrile-d3 σ(ppm): 1.9 (dd, 2H), 2.55(dd, 2H), 2.9 (s,3H), 3.29 (s,3H), 3.95(dd, 4H), 3.85 (s, 1H).

### EXAMPLE 2

### Tiotropium bromide

1.6 g (5 mmol) methylscopinium hexafluorophosphate (Example 1) and 2.0 g (7.8 mmol) methyl dithienylglycolate are refluxed in 50 ml acetone and in the presence of 10 g molecular sieve 4 Å for 50-70 hours.

The reaction mixture is filtered and the filtrate is combined with a solution of 0.3 g LiBr in 10 ml acetone. The unreacted N-methylscopinium bromide that crystallises out is separated off by filtration. After the addition of another 0.6 g LiBr (dissolved in acetone) tiotropium bromide is precipitated in an isolated yield of 30% (based on the compound according to Example 1 used).

### EXAMPLE 3

### Tiotropium hexafluorophosphate

Tiotropium hexafluorophosphate is not isolated within the scope of the reaction according to Example 2 but further reacted directly to form the tiotropium bromide.

For the purposes of characterising the tiotropium hexafluorophosphate this was prepared specifically and isolated. The following characterising data were obtained.

M.p.: 233-236° C. (melting with discoloration)

H-NMR: in acetone-d6: σ(ppm): 2.08 (dd, 2H), 2.23(dd, 2H), 3.32 (s, 3H), 3.50 (s, 3H), 3.62(s,2H), 4.28(m, 2H), 5.39(m, 1H) .6.25 (s), 7.02(m,2H), 7.027.22(m,2H), 7.46(m,2H), P-NMR: in acetone-d6: σ(ppm): −143.04, heptet, J=4.37.

### EXAMPLE 4

### Tiotropium bromide

31.5 g (100 mmol) methylscopinium hexafluorophosphate (Example 1) and 25.4 g (100 mmol) methyl dithienylglycolate are refluxed in 400 ml acetone and in the presence of 40 g of powdered molecular sieve 4 Å (Fluka) and DMAP (4,4-dimethylaminopyridine) for 24 h. (Molecular sieve was replaced by the same amount after 3 h.)

The reaction mixture is filtered, washed with 200 ml acetone, the filtrate is combined stepwise with a solution of 9.6 g LiBr (110 mmol) in 110 ml acetone. The unreacted N-methylscopinium bromide that crystallises out is separated off by filtration. (Fractionated precipitation). The crystal fractions were filtered off and dried. The composition of the fractions was determined by thin layer chromatography. Tiotropium bromide in an isolated yield of 16.6 g (35%) (based on the compound of Example 1 used). Purity HPLC>99%. Purity according to TLC: no impurities could be detected.

### EXAMPLE 5

### Tiotropium bromide

1.6 g (5 mmol) of methylscopinium hexafluorophosphate (Example 1) and 1.25 g (5 mmol) of methyl dithienylglycolate are stirred in 50 ml acetone and in the presence of 2 g of powdered molecular sieve 4 Å (Fluka) and 6 mg of potassium-tert.-butoxide at 0° C. for 4 h. The reaction mixture is filtered, washed with 20 ml acetone, the filtrate is combined stepwise with a solution of 0.7 g LiBr (13 mmol) in 11 ml acetone. The unreacted that crystallises out is separated off by filtration. (Fractionated precipitation). The crystal fractions were filtered off and dried. The composition of the fractions was determined by thin layer chromatography. The tiotropium bromide fractions were suction filtered, washed with acetone, recrystallised from water, washed with acetone and dried. 1.2 g of tiotropium bromide (48% yield based on the compound of Example 1 used) were isolated in this way. Purity HPLC: 99.8%. Purity according to TLC: no impurities were visible.

### EXAMPLE 6

### Tiotropium bromide

31.5 g (0.1 mol) methylscopinium hexafluorophosphate (Example 1) and 30.5 g (0.10 mol) of 2,2′-methyl dithienylglycolate are dissolved in 400 ml acetone and stirred in the presence of 90 g of zeolite of type 4 Å (Na₁₂Al₁₂Si₁₂O₄₈×n H₂O) and 0.2 g (1 mmol) potassium-tert.-butoxide over a period of 20-24 hours at 0° C.

The reaction mixture is filtered, the filtrate is combined with a solution of 8.7 g of LiBr (8.7 g 0.10 mol in 100 ml acetone).

The product that crystallises out is separated off by filtration, washed with acetone and then dried.

A yield of 41.4 g (87.7%) is obtained, with a 90% conversion level.

### EXAMPLE 7

### N-methylscopinium tetraphenylboranate

20 g (80 mmol) methylscopinium bromide are dissolved in 500 ml of methanol. 27.38 (80 mmol) sodium tetraphenylboranate, dissolved in 150 ml of methanol, are metered in. The resulting suspension is stirred for 10 min at ambient temperature and filtered.

The crystals separated off are washed with 50 ml of methanol and dried.

Yield: 39.1 g (91.73% yield); M.p.: 261° C.

### EXAMPLE 8

### Tiotropium tetraphenylboranate

0.245 g (0.5 mmol) methylscopinium tetraphenylboranate (Example 7), and 0.154 g (0.6 mmol) 2,2-methyl dithienylglycolate are dissolved in 25 ml acetone and stirred at 0° C. over a period of 20-30 hours in the presence of 1.0 g zeolite of type 4 Å (Na₁₂Al₁₂Si₁₂O₄₈×n H₂O) and 5 mg potassium-tert.-butoxide.

According to HPLC, after 26 hours 79% of the 2,2-methyl dithienylglycolate reacted have been converted into tiotropium tetraphenylboranate. (Non-isolated yield: 43%).

The reactions mentioned by way of example take place with virtually no formation of by-products.

1) Process for preparing scopinium salts of formula 1 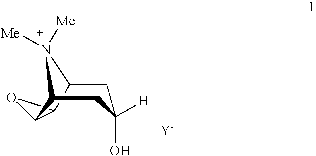
wherein
Y⁻ denotes a lipophilic anion with a single negative charge, preferably an anion selected from among hexafluorophosphate, tetrafluoroboranate, tetraphenylboranate and saccharinate, particularly preferably hexafluorophosphate or tetraphenylboranate
characterised in that a compound of formula 2 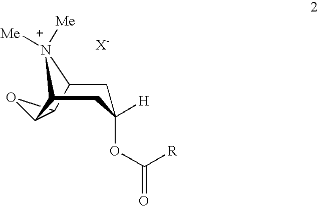
wherein
X⁻ denotes an anion with a single negative charge selected from among chloride, bromide, iodide, methanesulphonate, p-toluenesulphonate, nitrate and trifluoromethanesulphonate, preferably chloride, bromide, iodide, methanesulphonate, nitrate or trifluoromethanesulphonate, particularly preferably chloride, bromide or methanesulphonate, particularly preferably bromide; and
R denotes a group selected from C₁-C₄-alkyl, C₂-C₆-alkenyl and C₁-C₄-alkylene-phenyl, which may be substituted in each case by hydroxy, hydroxymethyl or C₁-C₄-alkoxy,
optionally in the form of the solvates or hydrates thereof, is saponified in a suitable solvent with the addition of a suitable base to form initially a compound of formula 3 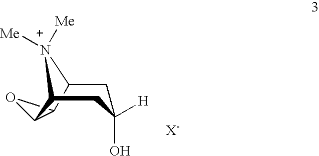
wherein X⁻ may have the meanings given above, and the compound of formula 3 without being isolated is converted into the compound of formula 1 by reacting with a salt Kat⁺Y⁻, wherein Kat⁺ denotes a cation selected from among Li⁺, Na⁺, K⁺, Mg²⁺, Ca²⁺, and Y⁻ may have the meanings given above.

2) Process according to claim 1, characterised in that the reaction is carried out with a compound of formula 2, wherein X⁻ may have the meanings given in claim 1 and
wherein
R denotes a group selected from —CH₃, —CH₂—CH₃, —CH₂—CH₂—OH, —CH(OH)—CH₃, —CH₂-phenyl, —CH(OH)-phenyl and —CH(CH₂OH)-phenyl, preferably —CH₃, —CH₂—CH₃, —CH₂-phenyl, and —CH(CH₂OH)-phenyl, particularly preferably —CH(CH₂OH)-phenyl.

3) Process according to claim 1,
wherein
Y⁻ denotes an anion with a single negative charge selected from among hexafluorophosphate, tetrafluoroboranate and tetraphenylboranate, preferably hexafluorophosphate.

4) Use of compounds of formula 2 according to claim 1 as starting compounds for preparing compounds of formula 1.

5) Process for preparing compounds of formula 1 of claim 1, characterised in that a compound of formula 2 is used as the starting compound.

6) Process for preparing tiotropium salts of formula 4 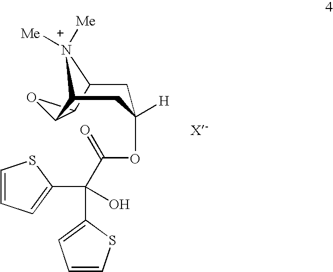
wherein
X′⁻ may denote an anion with a single negative charge, preferably an anion selected from among chloride, bromide, iodide, sulphate, phosphate, methanesulphonate, nitrate, maleate, acetate, citrate, fumarate, tartrate, oxalate, succinate, benzoate, p-toluenesulphonate and trifluoromethanesulphonate,
characterised in that a compound of formula 2 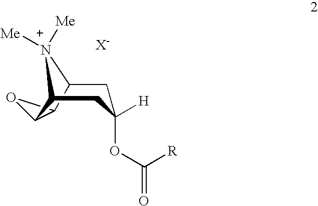
wherein
X⁻ denotes an anion with a single negative charge selected from among chloride, bromide, iodide, methanesulphonate, p-toluenesulphonate, nitrate and trifluoromethanesulphonate, preferably chloride, bromide, iodide, methanesulphonate, nitrate or trifluoromethanesulphonate, particularly preferably chloride, bromide or methanesulphonate, particularly preferably bromide; and
R denotes a group selected from C₁-C₄-alkyl, C₂-C₆-alkenyl and C₁-C₄-alkylene-phenyl, which may be substituted in each case by hydroxy, hydroxymethyl or C₁-C₄-alkoxy,
optionally in the form of the solvates or hydrates thereof, is saponified in a suitable solvent with the addition of a suitable base to form initially a compound of formula 3 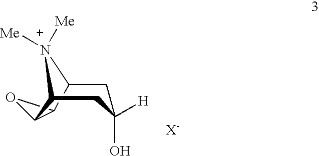
wherein X⁻ may have the meanings given above, and the compound of formula 3 without being isolated is converted, by reaction with a salt Kat⁺Y⁻, wherein Kat⁺ denotes a cation selected from among Li⁺, Na⁺, K⁺, Mg²⁺, Ca²⁺, and
Y⁻ denotes a lipophilic anion with a single negative charge, preferably an anion selected from among hexafluorophosphate, tetrafluoroboranate, tetraphenylboranate and saccharinate, particularly preferably hexafluorophosphate or tetraphenylboranate into the compound of formula 1 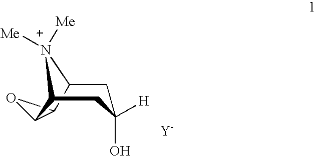
wherein Y⁻ may have the meanings given above, and then the compound of formula 1 is reacted in one step with a compound of formula 5 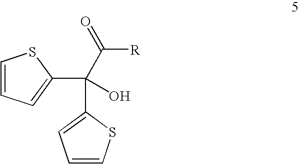
wherein
R denotes a group selected from among methoxy, ethoxy, propoxy, isopropoxy, isopropenyloxy, butoxy, O-N-succinimide, O-N-phthalimide, phenyloxy, nitrophenyloxy, fluorophenyloxy, pentafluorophenyloxy, vinyloxy, 2-allyloxy, -S-methyl, -S-ethyl and -S-phenyl,
in a suitable solvent to form a compound of formula 6 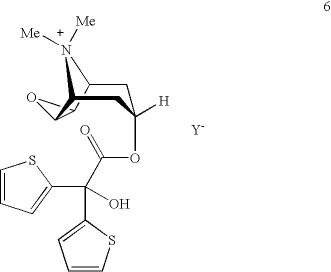
wherein the group Y⁻ may have the meanings given above, and the compound of formula 6 without being isolated is converted into the compound of formula 4 by reaction with a salt Kat⁺X′⁻, wherein Kat⁺ denotes a cation selected from among Li⁺, Na⁺, K⁺, Mg²⁺, Ca²⁺, organic cations with quaternary N and X′⁻ may have the meanings given above.

7) Use of compounds of formula 2 as starting compounds for preparing compounds of formula 4 of claim 6.

8) Use of compounds of formula 2 as starting compounds for preparing compounds of formula 6 of claim 6.
